(19) United States
(12) Patent Application Publication
(10) Pub. No.: US 2003/0032967 A1
(43) Pub. Date: Feb. 13, 2003

(54) Anastomotic device

(75) Inventors: Adrian Edward Park, Nicholasville, KY (US); Charles Francis Knapp, Georgetown, KY (US); William Edward Charish, Sudbury, MA (US); Hua Cui, Lexington, KY (US)

Correspondence Address: STEPTOE & JOHNSON LLP, 1330 Connecticut Avenue, N.W., Washington, DC, 20036, US

(73) Assignee: ParK Medical, LLC, Nicholasville, KY (US)

(21) Appl. No.: 10/175,159

(22) Filed: Jun. 20, 2002

Related U.S. Application Data
(60) Provisional application No. 60/299,618, filed on Jun. 20, 2001.

(51) Int. Cl. A61B 17/08

(52) U.S. Cl. 606/153

(57) ABSTRACT

The present invention is directed to gastrointestinal or enteric (including biliary) anastomosis and the like. The anastomotic device of the invention is a three dimensional woven tube of wire preferably formed from a thermal, smart memory metal. The outer loops or ends of the tube fold or loop back on deployment in a manner which holds the luminal interface of the anastomotic site into apposition at the deployment site. The woven tube is deployed using a canula with a retractable outer sleeve.

The present application claims the benefit of provisional application Serial No. 60/299,618, filed Jun. 20, 2001.

### BACKGROUND OF THE INVENTION

1. Field of the Invention

The present invention is directed to gastrointestinal and enteric (including biliary) anastomoses and the like. The woven tube of wire of the invention is a three dimensional structure wherein the outer loops or ends of the woven tube fold or loop back in a manner which holds the luminal interface of the anastomotic site into apposition at the deployment site.

2. Description of the Related Art

Surgical procedures often require the joining (anastamosis) of two vessels or hollow vicera. For example, a permanent anastomosis between the stomach and intestine may be required in the performance of gastric bypass surgery for the morbidly obese as well as to alleviate blockage in the common bile duct by draining bile from the duct to the small intestine during surgery for pancreatic cancer. Surgical anastomosis generally involves manual suturing of the two structures. This process can be technically demanding and time consuming. This complex surgical procedure is even more challenging during minimally invasive surgery (MIS) where the surgeon is required to use instruments that are poorly designed for this task.

### SUMMARY OF THE INVENTION

The present invention is directed to a woven tube of wire for use in an automated anastomotic delivery device for surgery with special emphasis on MIS. The primary component is the woven tube of wire which deforms to make an anastomotic device when inserted into the walls of two adjacent vessels or lumens. The use of such a device for joining (anastomosing) two gastrointestinal or enteric (including biliary) vessels or lumens or the like is new.

The anastomotic delivery device is designed to allow the wire mesh tube to be slipped over a canula and pulled longitudinally, causing the tube to become longer and very small in diameter. After the wire mesh tube is loaded onto the canula, an outer sleeve is pushed over the tube up to the streamlined end of the delivery device, thereby providing a smooth surface for inserting into a vessel or lumen in the body. After the loaded canula is inserted into the appropriate vessel or lumen, a small sharp pointed wire, initially retracted in the center of the canula, is exposed at the tip (such as by pushing on a button in the handle) in order to assist the surgeon when passing the canula through the walls of the vessels or lumens. Once the canula/sleeve has penetrated both walls and is properly positioned, the outer sleeve is retracted. The wire mesh tube is constructed from a thermal, shape memory alloy such as nitinol such that when the sleeve is retracted, heat from the body causes the wire mesh tube to contract longitudinally to produce the anastomosis. This design eliminates the necessity for using a mechanical compression component in the delivery system and, therefore, reduces the complexity and size of the delivery system. Sufficient force is applied to the wall tissues such that the holes between the two lumens is enlarged (for drainage) and leakage outside the two lumens does not occur.

Additional objects, advantages and other novel features of the invention will be set forth in part in the description that follows and in part will become apparent to those skilled in the art upon examination of the foregoing or may be learned with the practice of the invention.

### BRIEF DESCRIPTION OF THE DRAWINGS

FIG. 1 is a front view of the woven tube of wire in its deployed form in which the outer loops or ends of the woven tube have deformed and everted to form petals which hold the walls of the lumens into apposition, the front and back petals of the deployed anastomotic device being shown as dark black and light gray lines, respectively.

FIG. 2 is a side view of the woven tube prior to being slipped over a canula of the delivery device.

FIG. 3A is a front view of the woven tube similar to FIG. 1 with the walls of the lumens being omitted.

FIG. 3B is a side view of the woven tube of FIG. 3A.

FIG. 4A shows the delivery device having the woven tube loaded and the sleeve pushed over the tube up to the end of the delivery device.

FIG. 4B shows the delivery device inserted into a body cavity to a predetermined puncture site and further shows the tip of a wire, initially retracted in the canula, passed through the walls of the lumens.

FIG. 4C shows the end of the delivery device passed through the walls of the lumens with the sheath partially retracted to expose the woven tube slipped over the canula.

FIG. 4D shows the woven tube positioned at the juncture of the opposing puncture holes in the tissue, the walls of the lumens being held in a predetermined position guided by the delivery device.

FIG. 4E shows the initial stage of deployment of the woven tube as the ends of the woven tube begin forming a petal configuration.

FIG. 4F shows the woven tube in its deployed, flattened form gripping the walls of the lumens.

FIG. 4G shows the delivery device being retracted from the body cavity through the opening in the flattened woven tube, the left-hand side portion of FIG. 4G showing an end view of the deployed woven tube in the same manner as shown in FIG. 1.

### DESCRIPTION OF THE PREFERRED EMBODIMENTS

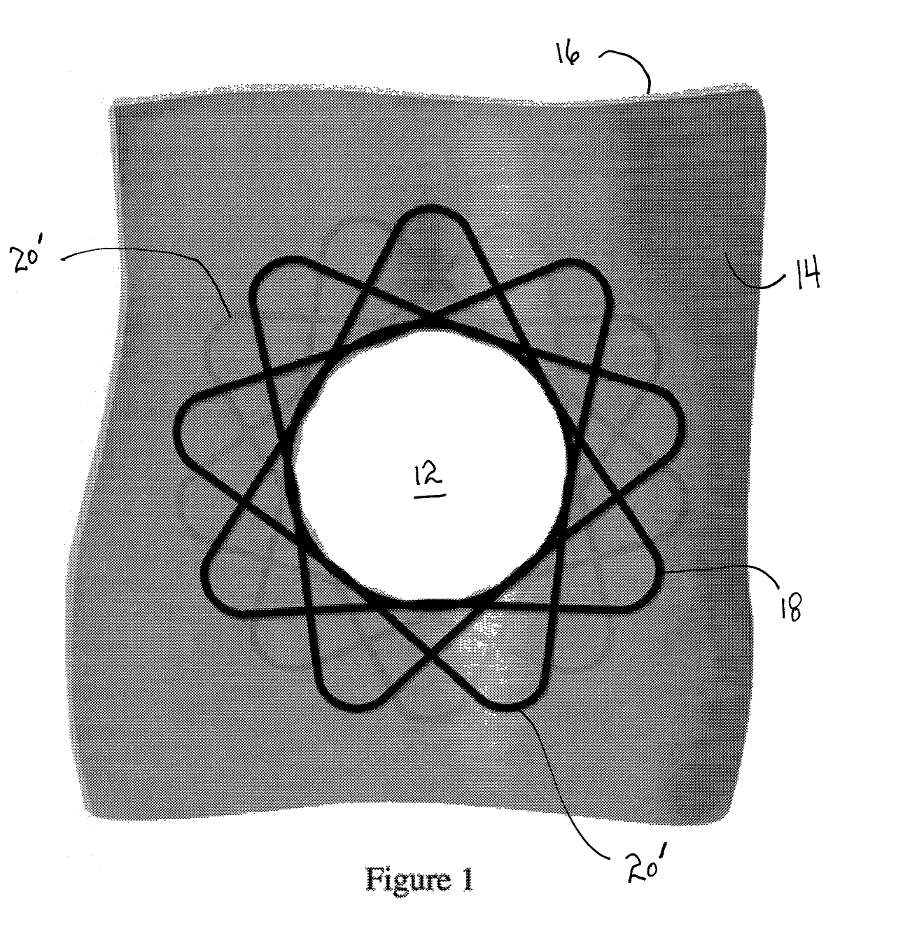

The tube 10 has an overlapping wire mesh design. The woven tube is designed to produce a round opening 12 between two layers of tissue 14, 16 and to hold the layers of tissue together for a watertight seal. The deployed anastomic device is essentially a woven tube 10 of wire 18 that is axially compressed as shown in FIG. 1.

The woven tube 10 is defined by the wire diameter, number of circumferential and longitudinal openings or diamonds 20, the tube length and the center diameter. The openings or diamonds 20′ at the longitudinal ends of the elongated woven tube are referred to as petals when the device is in the deployed shape (see FIG. 1).

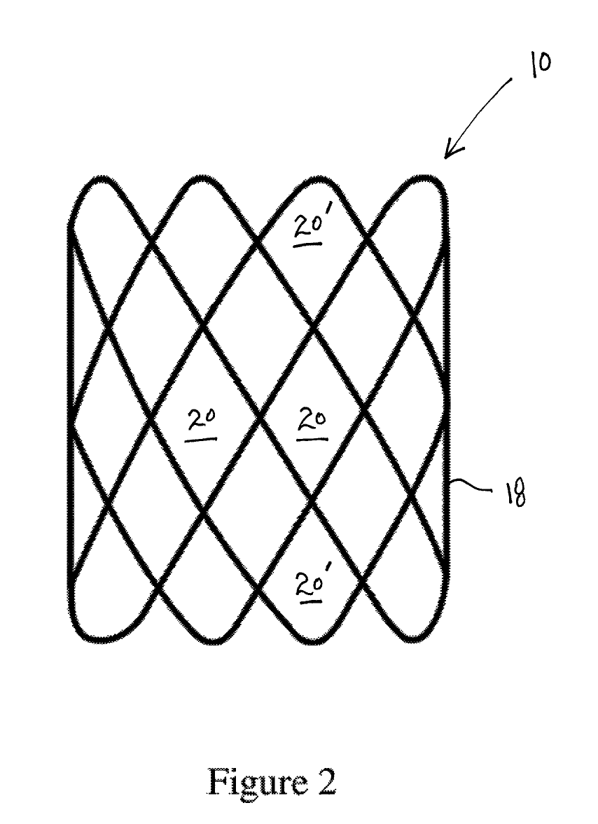

In use, the woven tube is forced into an elongated form (with much smaller diameter than that shown in FIG. 2), placed through openings between the wall tissues of two lumens and allowed to return to the flattened shape of FIG. 1. In the process, the tissues of both lumen walls are compressed between the petals of the flattened tube (see FIG. 1) with the center diameter 12 of the flattened tube forming an opening between the lumens.

The woven tube can be applied, for example, through the common bile duct, and pushed through so that it connects the duct to the jejunum. After the connection has been made, the tube can be caused to deform and evert so that the ends spread out like the petals of a flower and form a connection between the two ducts. Since the tube is made of a wire mesh, scar tissue will grow around the flattened tube and eventually form a permanent connection.

The woven tube is made out of a shape memory metal. A shape memory metal is an alloy that changes its plasticity as heat is applied, allowing it to change shape. If a shape memory metal is annealed in a desired form (in a longitudinally compressed form), after it is reshaped (in a cylindrical tube form) it will return to its annealed shape (flattened form) if it is reheated at a significantly lower temperature. The very special property of thermal memory is especially helpful in the design of a low profile and flexible delivery system. The preferred shape memory metal is a titanium-nickel alloy, most preferably a nearly equiatomic alloy of titanium and nickel called nitinol. Specific nitinol alloys, which also have superelastic properties, can reshape at body temperature.

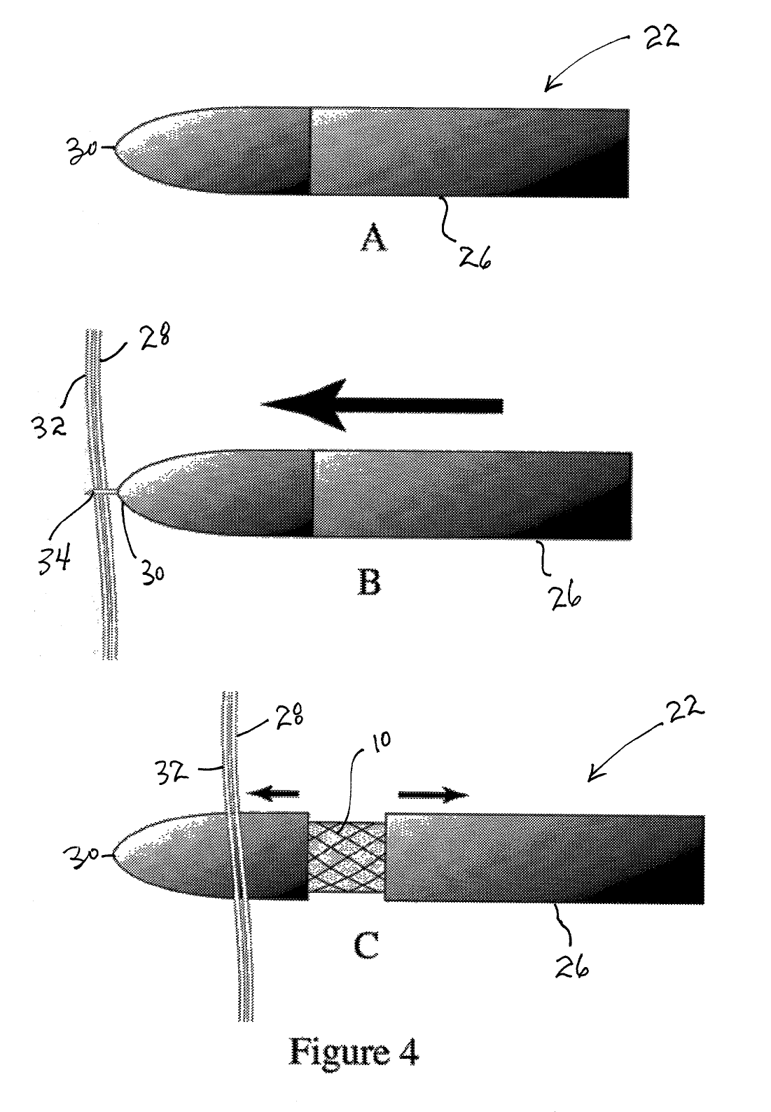

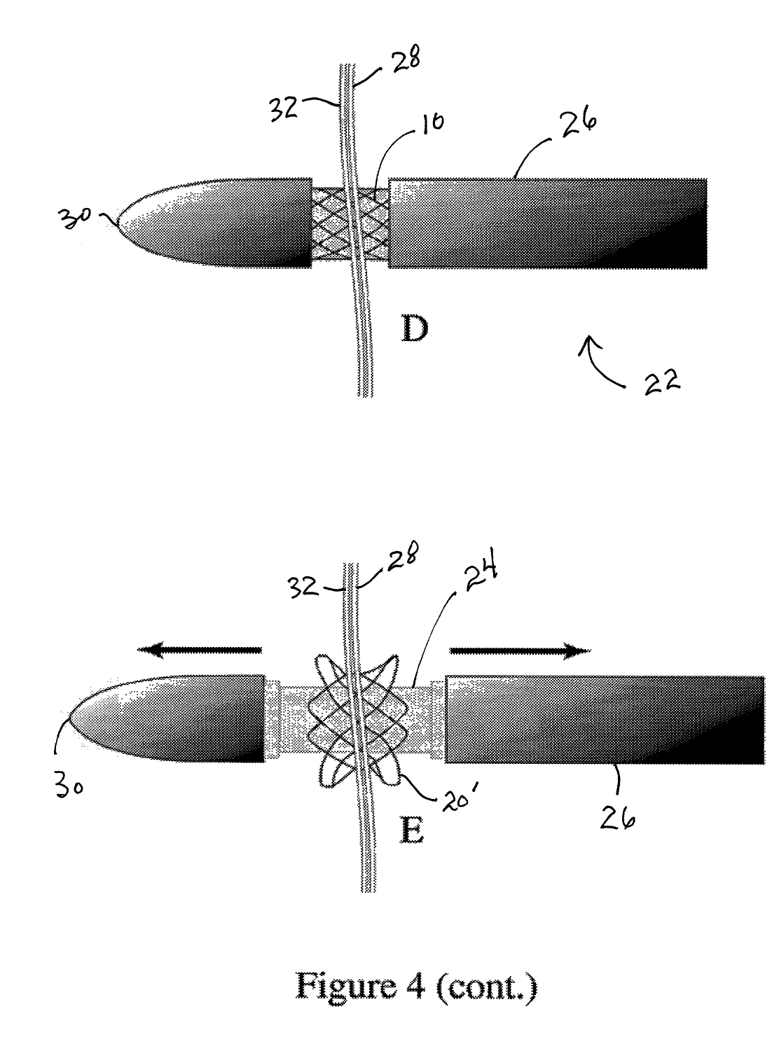

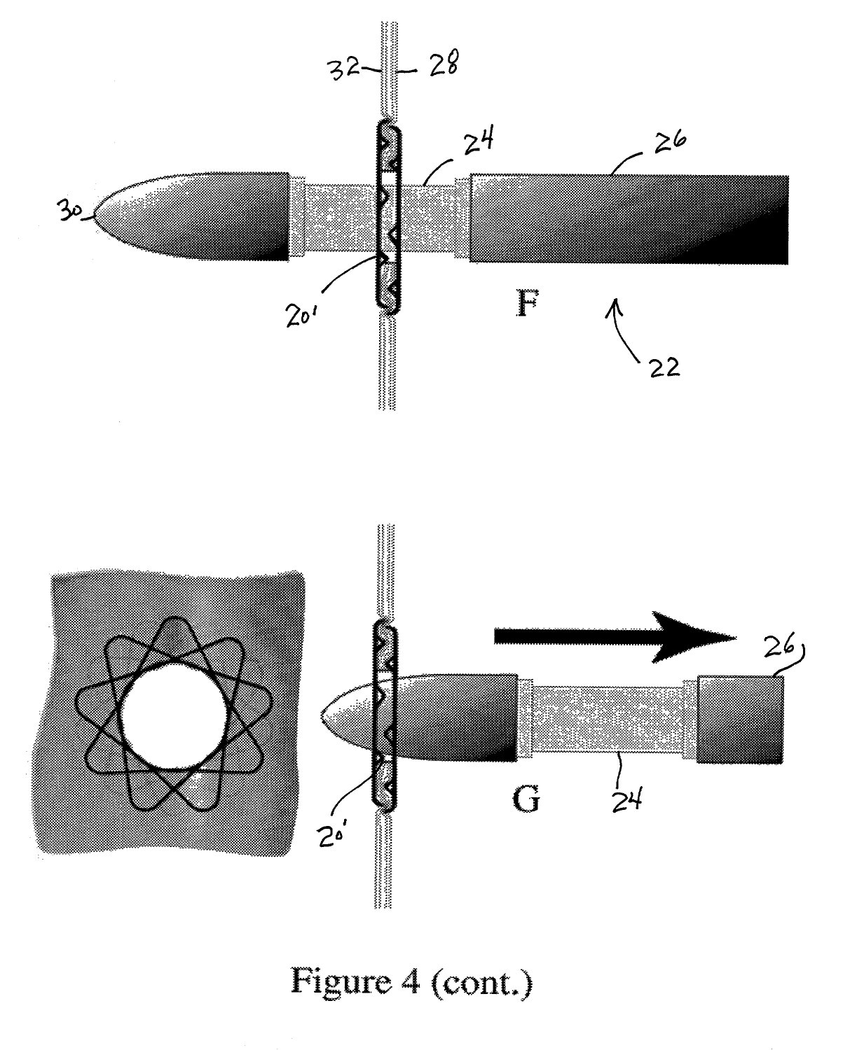

One embodiment of the delivery device 22 of the invention comprises a woven tube 10 mounted on a canula or delivery rod 24 covered by a retractable sheath 26 as shown in FIG. 4A. In use in a side-to-side intestinal anastomosis, for example, the delivery device 22 is inserted into the body cavity through a trochar or tube (not shown) and the end 30 of the delivery device 22 is positioned at a predetermined puncture site in a first intestinal segment 28 either proximal or distal to the desired anastomotic site and the delivery device 22 is advanced intraluminally to the anastomotic site.

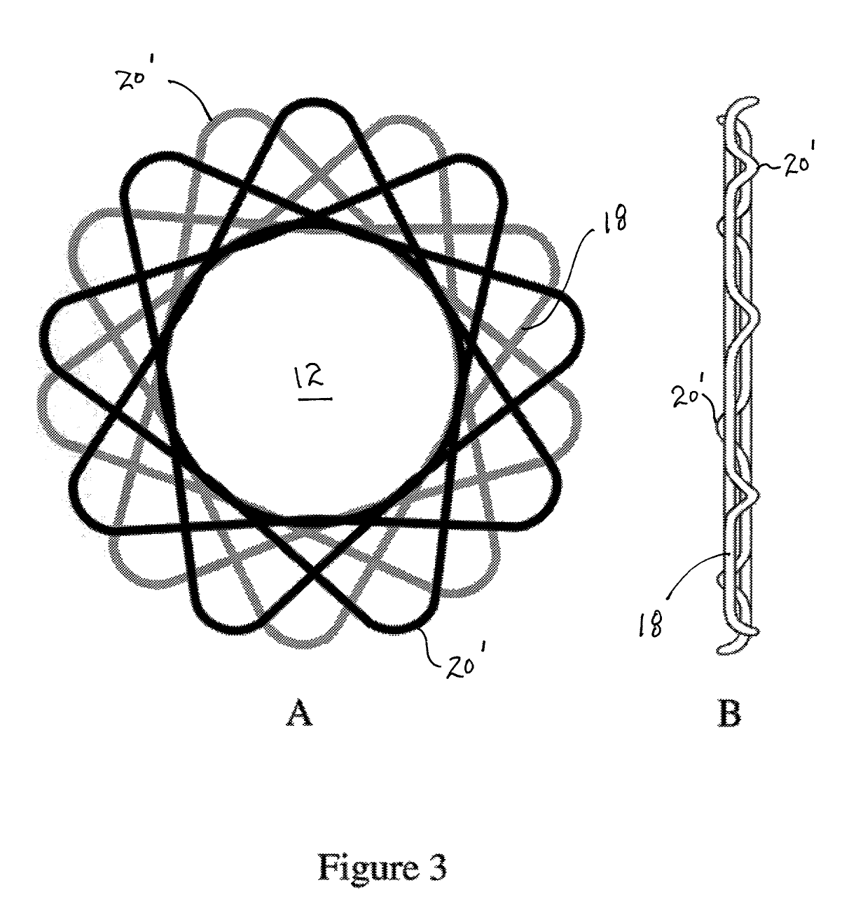

The second intestinal segment 32 is brought into close apposition to the first segment at the anastomotic site and the sharp tip of a wire 34, initially retracted in the center of the canula 24, is used to pierce through the wall of the first segment 28 and the wall of the second segment 32 and into the lumen of the second segment as shown in FIG. 4B. The sheath 26 is retracted and the woven tube 10 is deployed as shown in the sequence of FIGS. 4C, 4D, 4E and 4F at the juncture of the apposing holes created by the tip of the wire 34 and assumes the petal configuration at the site to hold the two pieces of intestine in apposition. The woven tube deployed through two layers of intestine is shown in FIGS. 4F and 4G. The opposed petals 20 on opposite sides of the two layers of intestine 28, 32 are preferably interdigitated as shown in FIGS. 1, 3A and B and 4G.

The foregoing description of a preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled.

We claim:

1. An anastomotic device, comprising a woven tube of wire constructed from a thermal, shape memory alloy having outer loops or ends which thermally deform and evert when inserted into walls of two adjacent lumens at a luminal interface of an anastomotic site, the ends of the tube thermally deforming and everting to form petals in a manner which holds the luminal interface of the anastomotic site into apposition.

2. The device of claim 1, wherein the thermal, shape memory alloy is a titanium-nickel alloy.

3. The device of claim 1, wherein the opposed petals are interdigitated.

4. An anastomotic delivery device, comprising a woven tube of wire, a canula having an end designed to allow the tube to be slipped over the canula and pulled longitudinally causing the tube to become longer and small in diameter, an outer sleeve adapted to be pushed over the tube up to the end of the device thereby providing a smooth surface for inserting through walls of lumens in a body, and subsequently retracted, a wire having a tip, initially retracted in the canula, and adapted to be exposed at the tip to assist a surgeon when passing the device through the walls of the lumens, the tube being constructed from a thermal, shape memory alloy such that when the sleeve is retracted, heat from the body causes the tube to contract longitudinally to produce the anastomosis.

5. The device of claim 4, wherein the thermal, shape memory alloy is a titanium-nickel alloy.

6. The device of claim 4, wherein the tube contracts longitudinally to cause ends of the tube to deform and evert to form petals to produce the anastomosis.

7. The device of claim 6, wherein the opposed petals are interdigitated.

8. A method of deploying an anastomosis delivery device, comprising mounting a woven tube of wire constructed from a thermal, shape memory alloy on a canula covered by a retractable sheath, inserting the device into a body cavity through a tube and to a predetermined puncture site of a first segment either proximal or distal to a desired anastomotic site, advancing the device intraluminally to the anastomotic site, bringing a second segment into close apposition to the first segment at the anastomotic site, piercing a wall of the first segment and a wall of the second segment and into a lumen of the second segment, retracting the sheath and deploying the tube at a juncture of apposing puncture holes created by the device, the ends of the tube forming a petal configuration at the anastomotic site to hold the two segments in apposition.

9. The method of claim 8, wherein the thermal, shape memory alloy is a titanium-nickel alloy.

10. The method of claim 8, wherein the first and second segments are intestinal segments.

11. The method of claim 8, wherein one of the first and second segments is a bile duct and the other of the first and second segments is the jejunum.

12. The method of claim 8, wherein the anastomosis is a side-to-side anastomosis.
